(19) United States
(12) Patent Application Publication
(10) Pub. No.: US 2006/0259121 A1
(43) Pub. Date: Nov. 16, 2006

(54) Device for inserting an object into the interior of a blood vessel or the heart

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/433,492

(22) Filed: May 12, 2006

(30) Foreign Application Priority Data
May 14, 2005 (DE) 102005022423.7-44

(51) Int. Cl. A61F 2/06

(52) U.S. Cl. 623/1.11

(57) ABSTRACT

A device (1) used for applying an object (2) in an interior of the body, for example, for inserting a closure into an undesired opening (3) between the left atrium and the right atrium of a heart (4) is provided. The device has a delivery catheter (5), whose distal end (5a) opens at a point, that is, it is adapted to be moved to the point, at which the object (2) is to be placed or applied. Typically, an insertion device is used to insert the catheter (5) from the outside into a blood vessel (6). So that no particles can become detached and enter into the bloodstream when the object (2) or closure is moved on the inside of the catheter (5), the device (1) has a receptacle (9) or sleeve for encompassing or holding the object (2), wherein the object (2) can be transported and moved by the catheter (5), without being brought into contact with the inside of the catheter or tube (5).

### BACKGROUND

The invention relates to a device for inserting and placing or applying an object in the interior of a blood vessel or the heart, for example, for delivering a stent or a mechanical blood filter or cage or a closure for an undesired opening between the left atrium and the right atrium of the heart, and includes a delivery catheter, which is formed essentially as a tube and whose distal end opens in the position of use at the point, where the object should be placed, and with an insertion device, with which the catheter can be inserted from the outside into a blood vessel.

Such devices are known from practice and have proven effective primarily in the cases, in which an ASD closure consisting, for example, of a nitinol wire mesh, must be inserted and placed in the heart. Here, it is typical that the delivery catheter is formed of a flexible plastic tube, through which the ASD closure is moved and brought to its designated location.

Here, it has been shown that there is a risk that the object, especially if it consists of a metal wire mesh, strips or rubs off particles that could enter into the bloodstream and into the heart when the object is moved on the inside of the plastic pipe.

From US 2002/0013599 A1, a comparable device is known, with which an expandable stent is to be inserted into a blood vessel, which is gripped by projections on the inside, which are arranged on a delivery element that can be moved in a catheter tube. Here, there is also the risk that this stent, which is exposed on the outside, will come into frictional contact with the delivery catheter and strip or rub off particles from the catheter.

### SUMMARY

Therefore, there is the objective of creating a device of the above-noted type, with which it is possible to prevent an object, for example, an ASD closure, from abrading and/or entraining particles from the inside of the catheter when it is fed through the delivery catheter.

To meet this objective, the previously known device is improved in that, within the catheter, there is a receptacle that is movable in the interior of the catheter or tube up to its distal end for the object, the receptacle is embodied to encompass the object or with a sleeve-like shape and uses its inner elongated cavity for housing the object during its transport to the distal end of the catheter, and the receptacle has at its distal end an opening or an output for pushing out or discharging the object from the receptacle.

In this way, the object to be inserted can be prevented from contacting the inside of the delivery catheter during its transport through the delivery catheter, so that it cannot strip particles from the catheter and also no particles can be abraded from the object itself. The receptacle can be moved as a transport vessel through the delivery catheter or through the catheter tube and comes into contact with the inside of the catheter, but does not reach into the body, so that even if particles should become detached by the receptacle moving within the catheter, there is no way at all for these particles to enter into the body, into the bloodstream, or into the heart.

Above all, a sleeve-shaped receptacle, which thus has a closed wall, is favorable, because the separation of the object to be inserted from the inside of the catheter tube is guaranteed particularly well in this way.

The catheter tube can be formed of metal and can be flexible. Therefore, parts or particles can be prevented to an even better degree from detaching from the inside of the catheter, because metal also has a high resistance against abrasion, so that the receptacle used as a transport vessel cannot detach particles even during the movement through this metallic catheter tube or the flexible metal pipe.

For good flexibility of the catheter it is useful when the catheter tube has slots, which run in the peripheral direction, which are spaced apart from each other, and which are arranged, in particular, parallel to each other, wherein they can be connected to form an approximately helical slot or embodied as individual slots. A catheter with such a configuration can be adapted especially well to a wide variety of bends or curvatures of blood vessels.

In this way, a sufficient or improved resistance of the catheter can be achieved, in that the slotted tube or metal tube has at least one stabilization strip, especially made from metal, which runs along the extent of the tube and which is connected, especially welded, to the tube. Here, an arrangement of this stabilization strip on the outer side of the catheter tube is preferred. This stabilization strip can prevent the catheter tube from possibly becoming deformed or elongated in the longitudinal direction especially in the case of slots, which can therefore deform the receptacle or the transport vessel moving through the tube in an undesired way.

For the simplest possible discharge of the transported object, at the distal end of the catheter tube there can be a stop extending radially inwards for the receptacle. This guarantees that the receptacle or the transport vessel forming the receptacle cannot be inadvertently pushed out of the catheter tube for the object to be applied.

Here, the stop can be formed as a projection or ring encompassing the end side or the inner periphery of the tube or the catheter. Thus, the receptacle is stopped reliably at this stop, so that then the object to be implanted or to be applied can be pushed from it, whereby it simultaneously also leaves the catheter tube.

The stop can be flush with the opening of the tube or the catheter or it can form this opening. Thus, the distal end of the receptacle is led as far as possible to the distal end of the catheter tube.

For the simplest possible discharge of the transported object, for moving the receptacle there can be a slide or obturator attaching directly or indirectly to the receptacle, and the slide or obturator can also be used preferably for discharging the object from the receptacle after reaching the stop.

Here, the obturator can be connected detachably or screwed to the object. Thus, the displacement process during the transport can be realized such that the obturator connected detachably to the object is pushed forward within the tube, whereby simultaneously the receptacle containing the object is pushed until it reaches the stop. A further pushing motion then causes the object to be pushed from the receptacle or from the transport vessel and thus also from the catheter tube. Here, it is favorable that the friction between the object and its receptacle is greater than that between this receptacle and the catheter tube.

So that the receptacle can also follow curves and bends easily, the sleeve acting as the receptacle can have a flexible wall.

Tests have shown that it is favorable when the preferably metal tube of the catheter and/or the especially sleeve-shaped receptacle has a wall thickness of approximately 1/10 mm or less or somewhat more. This produces a reduced outer dimension of the catheter tube relative to a plastic tube or permits a greater inner cross section, in order to be able to transport a correspondingly larger object.

If necessary, special measures can also be taken to keep the friction between the especially sleeve-shaped receptacle and the catheter sleeve less than that between the object and the receptacle. However, because the object is usually compressed in the receptacle, there is already a greater resistance of this object against it being pushed out from the receptacle relative to the resistance acting on the receptacle when moving within the catheter tube. Thus, usually no special measures are needed to adequately hold the object in the sleeve-shaped receptacle until this has reached the distal end of the catheter tube.

### BRIEF DESCRIPTION OF THE DRAWINGS

Below, preferred embodiments of the invention are described in more detail with reference to the drawing. Shown in partially schematic representation are:

FIG. 1 is a view of a delivery catheter, which is inserted into a blood vessel and pushed forward into a region of the atria of the heart and with which an object can be inserted for closing an undesired opening between the left atrium and the right atrium of the heart,

FIG. 2 is a longitudinal section view at an enlarged scale of the heart with an undesired opening in the septum between the left atrium and the right atrium, after an ASD closure is inserted into this opening, with this closure having been fed through the catheter tube and still being connected to an obturator or pusher used for pushing the closure out of the tube,

FIG. 3 is a side view of the catheter, at whose proximal end the obturator projects,

FIG. 4 is a view, at an enlarged scale, of the distal end of the catheter tube marked in FIG. 3 by a circle, shown in longitudinal section, wherein in the interior of the catheter tube a movable receptacle as a transport vessel is visible, which contains the object to be placed in compressed form,

FIG. 5 is a view corresponding to FIG. 4, wherein the receptacle has reached a stop, which is located at the distal end of the delivery catheter and which has an annular shape and an inner opening, which is approximately flush with the inner opening of the receptacle, so that the object can be pushed with the help of the obturator—as shown—through further forward movement of the obturator until the object has reached the position shown in FIG. 2 and there forms an ASD closure, for example, due to its production from memory material,

FIG. 6 is a view of the obturator or pusher with the object to be transported,

FIG. 7 is an enlarged scale view of the obturator, the object to be transported, and their mutual detachable connection with the help of a thread, wherein the object is arranged in the receptacle, which can move within the guide catheter or delivery catheter,

FIG. 8 is a first side view of a delivery catheter, which, for increasing its flexibility, is provided with parallel, preferably helical slots and which is stabilized on the outside by a strip running in the direction of the longitudinal extent,

FIG. 9 is a second side view of the delivery catheter of FIG. 8,

FIG. 10 is an enlarged scale, cross-sectional view of the delivery catheter according to FIGS. 8 and 9,

FIG. 11 is a first side view of a modified embodiment of the delivery catheter, which is provided with parallel slots extending approximately in the peripheral direction perpendicular to its longitudinal center axis, wherein the sections of the delivery catheter separated from each other by these slots are connected by at least one stabilization strip extending in the direction of the longitudinal extent and connected or welded to the catheter

FIG. 12 is a second side view of the embodiment of the delivery catheter of FIG. 11, and

FIG. 13 is an enlarged scale, a cross-sectional view of the delivery catheter according to FIGS. 11 and 12.

### DETAILED DESCRIPTION OF THE PREFERRED EMBODIMENTS

A device designated as a whole with 1 is used for inserting and placing or applying an object 2, which in one preferred embodiment is a closure for an undesired opening 3 between the left atrium and the right atrium of the heart 4, but can also be used for placing or applying a different object in the interior of a blood vessel, for example, for delivering a stent or a mechanical blood filter or cage or the like.

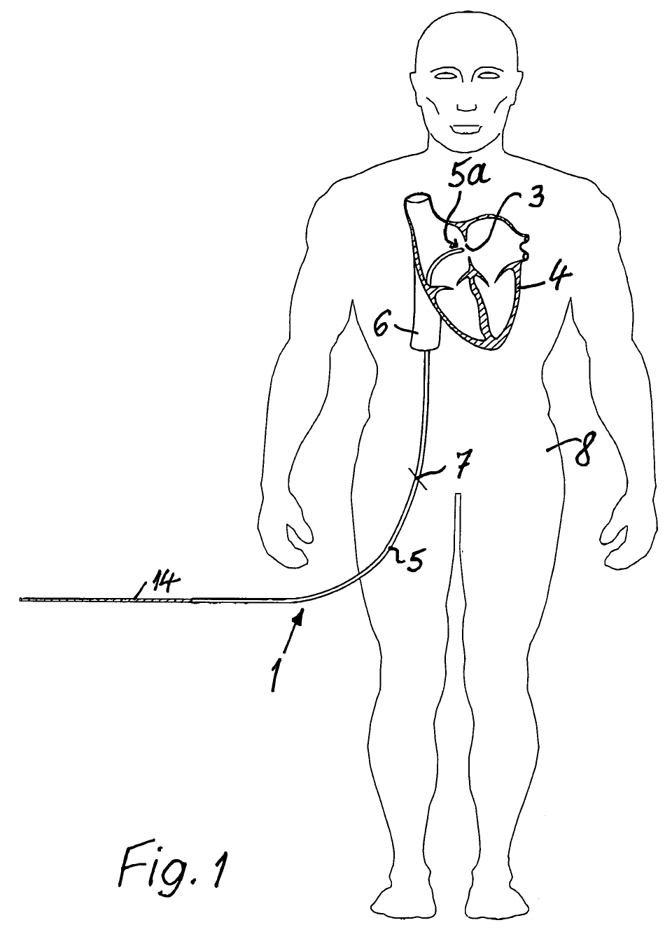

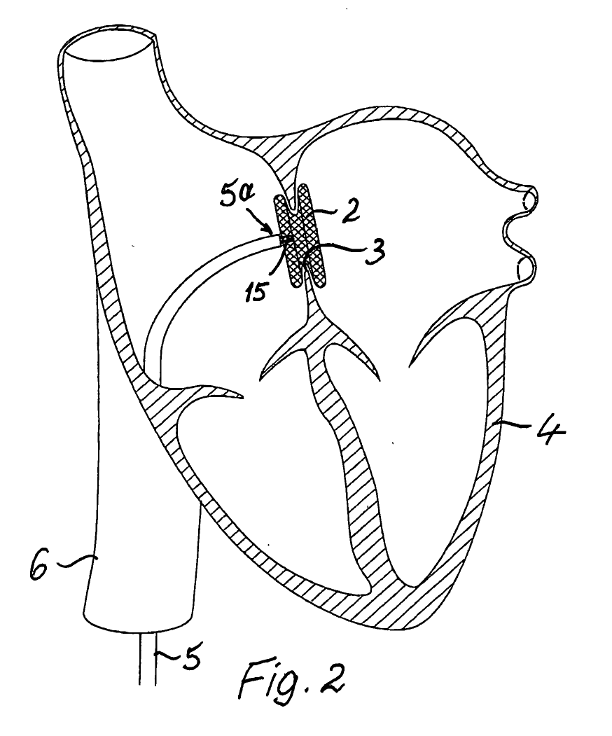

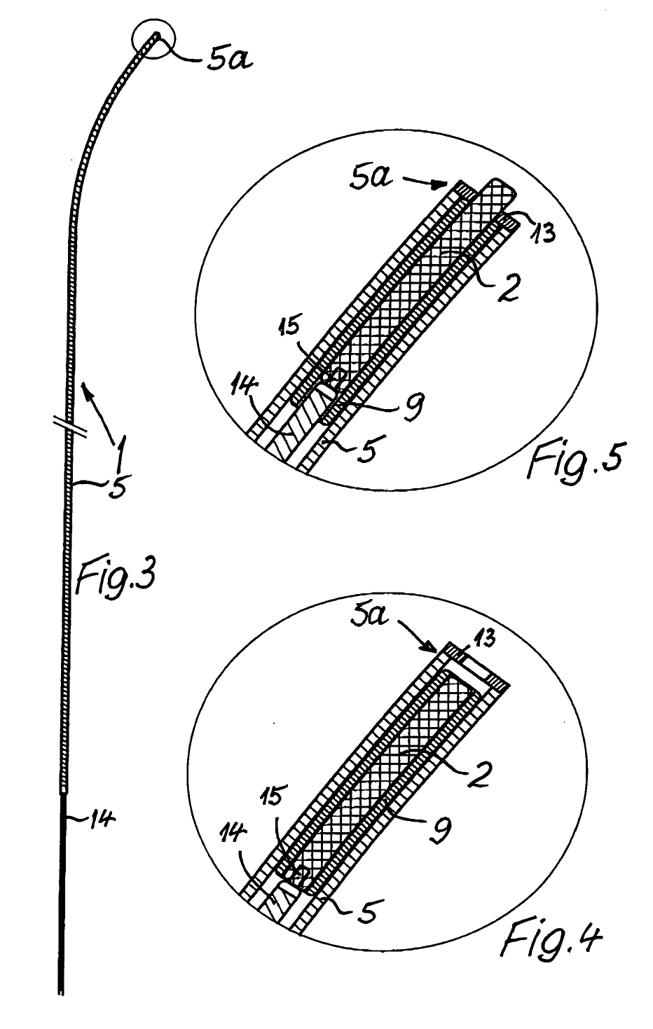

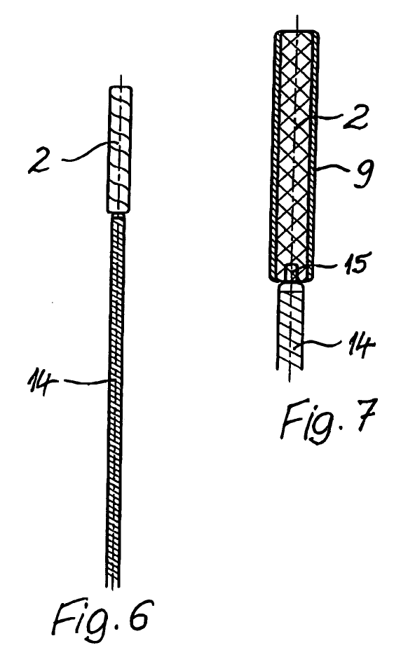

An essential part of the device 1 is a delivery catheter 5, which is formed essentially as a tube or optionally as a pipe, and includes a distal end 5a, which as shown in FIG. 2, opens at a point in the position of use where the object 2 is to be placed and applied. Furthermore, the device 1 includes an insertion device, which is not shown in the figures and with which the delivery catheter 5 can be inserted from the outside into a blood vessel 6 leading to the heart 4. In FIG. 1, a point 7 on a human body 8 where the catheter 5 can be inserted in an appropriate blood vessel 6 can be seen. In a known way, at this point a corresponding insertion device can be provided.

In FIGS. 4 and 5, it is shown that in the catheter 5, also designated below as “tube 5”, there is a receptacle 9 for the object 2, which receives the object 2 completely and encompasses it on the longitudinal sides, as FIGS. 4 and 7 show.

According to FIGS. 4 and 5, the receptacle 9 can move in the interior of the catheter or tube 5 up to its distal end 5a, that is, the receptacle 9 is used as a transport means or transport vessel for the object 2, for example, an ASD closure, as shown in FIG. 2.

At its distal end, the receptacle 9 has an opening or an outlet for pushing out or discharging the object 2, as shown in FIG. 5. Thus, the object 2 can be pushed within the receptacle 9 through the delivery catheter 5 up to its distal end 5a, without itself coming in contact with the inside of the delivery catheter 5. Thus, no particles can be abraded from this inside of the tube or catheter 5 by the object 2 and led into the bloodstream.

Here, the receptacle 9 is formed as a sleeve with a closed wall for encompassing the object 2, so that a compressed object 2 also remains completely separated from the inside of the guide catheter 5. The sleeve acting as a receptacle 9 and its inner longitudinal hole are used for housing the object 2 during its transport to the distal end 5a of the catheter 5.

The catheter tube 5 can be formed of metal and can have a flexible form.

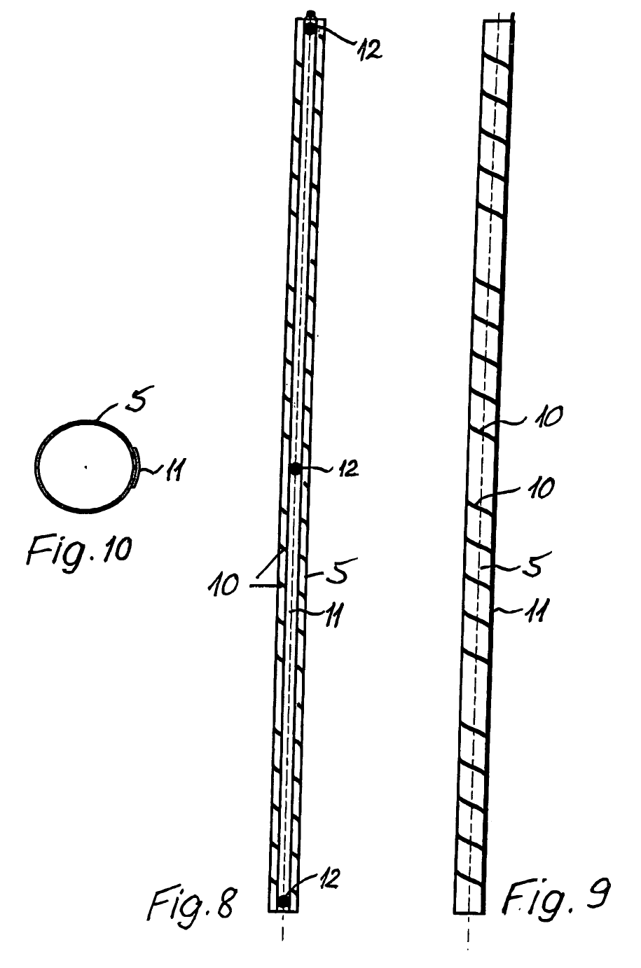

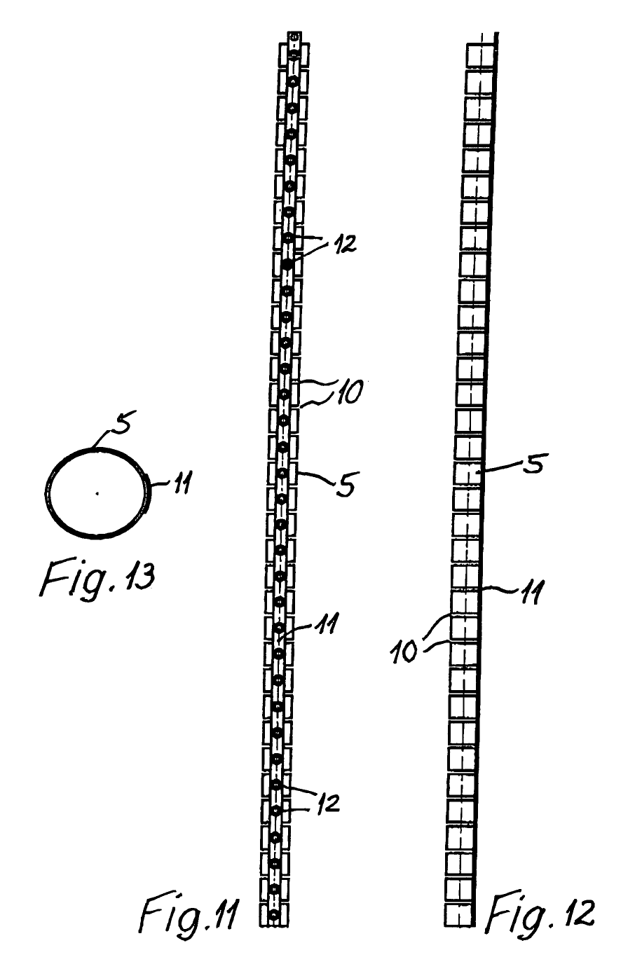

In the embodiments according to FIGS. 8 to 13, the catheter tube 5 has slots 10, which extend in the peripheral direction, which are spaced apart from each other, and which are arranged or extend parallel to each other in both embodiments (FIGS. 8 to 10 on one hand and FIGS. 11 to 13 on the other hand) .Here, in FIGS. 8 and 9, the slots 10 can be connected to form one approximately helical slot. Alternatively, the slots 10 are formed as individual slots according to FIGS. 11 to 13. In each case, greater flexibility of the catheter or tube 5 is produced especially when it is formed of metal.

So that the slotted catheter 5 cannot become elongated due to the sleeve-shaped receptacle 9 and its displacement in the longitudinal direction, the slotted tube 5 has a preferably metal stabilization strip 11, which is connected, and more preferably welded, to the tube 5 in the embodiments along its extent.

In FIG. 8, three welding points 12 can be seen over the entire length, while according to FIG. 11 each individual section of the tube 5 is connected to the stabilization strip 11 via such a welding point 12, because the slots 10 run over the entire periphery of the catheter 5.

In FIGS. 4 and 5, a radially inwardly extending stop 13 for the receptacle 9 is provided at the distal end 5a of the catheter tube 5. According to FIG. 5, the receptacle 9 is prevented from having its distal end being pushed out from the distal end 5a of the catheter 5 at the stop 13, so that in a further pushing process, the object 2 can be pushed both from the receptacle 9 and also from the catheter 5.

Here, the stop 13 is formed as a circular ring on the end of the tube or catheter 5 and whose inner opening matches the inner cross section of the sleeve-shaped receptacle 2, so that the object 2 can move smoothly and unimpaired through this annular stop 13. Here, the stop 13 practically forms the opening of the tube or catheter 5 and is fixed on the end of the catheter 5.

For moving the receptacle 9, there is a slide or obturator 14, which attaches indirectly to the receptacle and which projects past the proximal end of the catheter 5 and thus enables a corresponding handling and pushing movement relative to the catheter 5. Here, the slide or obturator 14 or pusher is also used for pushing the object 2 from the receptacle 9, after it has reached the stop 13, as shown primarily in FIG. 5.

Here, the object 2 according to FIG. 7 and also according to FIGS. 4 and 5 is initially connected detachably to the obturator 14, for example, by means of a threaded connection 15. Therefore, it is possible to push the object 2 according to FIG. 5 and move it up to the point where it is to be placed, thus, for example, in an undesired opening 3 between the left atrium and the right atrium of the heart 4, where the object 2 can open up from its initially compressed shape limited by the receptacle or the sleeve 9 into its final desired shape as a closure, which then closes the opening 3. Then the threaded connection 15 between the obturator 14 and the object 2 can be disconnected, when the object has reached, namely, its exactly desired position, so that the obturator or pusher 14 can then be withdrawn.

It should also be mentioned that the sleeve acting as the receptacle 9 has on its side a flexible wall and the, for example, metal tube or catheter 5 and also the sleeve-shaped receptacle 9 can each have a wall thickness of somewhat less than or somewhat more than or also exactly 1/10 mm, which produces a space-saving arrangement.

It was already mentioned that the obturator or pusher 14 can attach indirectly to the receptacle 9, in that it attaches, namely, to the object 2 located in the receptacle 9, wherein, nevertheless, initially also the receptacle 9 is pushed until it reaches the stop 13, because the friction between the sleeve-shaped receptacle 9 and the catheter tube 5 is less than the friction between the object 2 and the receptacle 9, in which the object 2 is also usually housed under a certain pressure.

However, it would also be possibly conceivable that a correspondingly shaped tube-like pusher attaches directly to the receptacle 9. In the interior of the pusher, another pusher or obturator can run for pushing out the object 2.

The device 1 is used for applying an object 2 in the interior of the body, for example, for inserting a closure in an undesired opening 3 between the left atrium and the right atrium of a heart 4. For this purpose, it has a delivery catheter 5, whose distal end 5a opens at the point, that is, it is moved to the point, at which the object 2 is to be placed or applied. Typically, an insertion device is used to insert the catheter 5 from the outside into a blood vessel 6. So that no particles can become detached and enter into the bloodstream when the object 2 or closure is moved on the inside of the catheter 5, the device 1 has a receptacle 9 or sleeve for encompassing or holding the object 2, wherein the object 2 can be transported and pushed by the catheter 5, without being brought into contact with the inside of the catheter or tube 5.

1. Device (1) for inserting and placing or applying an object (2) in an interior of a blood vessel or heart, comprising a delivery catheter (5), formed generally as a tube and having a distal end (5a) that is adapted to be positioned at a point where the object (2) is to be placed, an insertion device, with which the catheter (5) can be inserted from an outside into a blood vessel (6), a receptacle (9) for the object (2), which can move in an interior of the catheter (5) up to the distal end (5a), the receptacle (9) is formed for encompassing the object (2) or has a sleeve shape and an inner elongated cavity thereof is used for housing the object (2) during its transport to the distal end (5a) of the catheter (5), and the receptacle (9) has on a distal end thereof an opening or an outlet for pushing or discharging the object (2) from the receptacle (9).

2. Device according to claim 1, wherein the catheter tube (5) is formed of metal and has a flexible form.

3. Device according to claim 1, wherein the catheter tube (5) has slots (10), which extend in a peripheral direction, and which are spaced apart from each other, and are arranged generally parallel to each other, wherein the slots are connected to form one approximately helical slot.

4. Device according to claim 3, wherein the slotted tube (5) has at least one stabilization strip (11), which extends along a length thereof is connected to the tube (5).

5. Device according to claim 1, further comprising a radially inwardly extending stop (13) for the receptacle (9) located at the distal end (5a) of the catheter tube (5).

6. Device according to claim 5, wherein the stop (13) is formed as a circular projection or ring on the end or on an inner periphery of the catheter (5).

7. Device according to claim 6, wherein the stop (13) is flush with the opening of the catheter (5) or forms the opening.

8. Device according to claim 5, wherein for moving the receptacle (9), a slide or obturator (14) is provided attached directly or indirectly to the receptacle and the slide or obturator (14) is also adapted for use to push the object (2) from the receptacle (9) after reaching the stop (13).

9. Device according to claim 8, wherein the slide or obturator (14) is connected detachably to the object (2).

10. Device according to claim 1, wherein the receptacle is formed of a sleeve which has a flexible wall.

11. Device according to claim 1, wherein the catheter tube (5) is metal and has a wall thickness less than or somewhat greater than or equal to approximately 1/10 mm.

12. Device according to claim 1, wherein friction between the receptacle (9) and the catheter tube (5) is less than that between the object (2) and the receptacle (9).

13. Device according to claim 1, wherein the object located in the receptacle comprises a stent, a mechanical blood filter, a cage or a closure for an undesired opening (3) between the left atrium and the right atrium of the heart (4).

14. Device according to claim 1, wherein the catheter tube (5) has slots (10), which extend in a peripheral direction, and which are spaced apart from each other, and are arranged generally parallel to each other, wherein the slots are formed as individual slots.

15. Device according to claim 1, wherein the receptacle (9) is sleeve-shaped and has a wall thickness less than or somewhat greater than or equal to approximately 1/10 mm.
